(19) United States
(12) Patent Application Publication
(10) Pub. No.: US 2002/0160037 A1
(43) Pub. Date: Oct. 31, 2002

(54) Self-adhesive wound dressings with adhesive wound management region

(76) Inventors: Helge Ahrens, Hamburg (DE); Michael Schink, Hamburg (DE); Jochen Kenndoff, Gawa Timur (ID); Gunther Sachau, Quickborn (DE); Reiner Leutz, Reinbek (DE)

Correspondence Address: Kurt G. Briscoe, Norris McLaughlin & Marcus, P.A., 220 East 42nd Street - 30th Floor, New York, NY, 10017, US

(21) Appl. No.: 09/955,852

(22) Filed: Sep. 19, 2001

(30) Foreign Application Priority Data
Sep. 22, 2000 (DE) 100 47 884.0

(51) Int. Cl. A61L 15/00

(52) U.S. Cl. 424/445

(57) ABSTRACT

A wound dressing comprising a moisture vapor pervious polyurethane matrix beveled from an especially central point of the wound dressing toward the edge.

This invention relates to a self-adhesive wound dressing which comprises an adhesive wound management region and is useful especially on pressure points, on blisters and open blisters on the heel, on the ball of the thumb and on the fingers.

Modern wound management products such as hydrocolloids (see for example “Hydrokolloide” by R. Lipmann in “Medical Device & Diagnostic Industry”, June 1999), which were developed for colostomy and professional wound management applications, are increasingly used.

Wound management products based on hydrocolloids have advantages over traditional plasters. They generate a moist wound healing medium which stops the wound drying out and creates an optimal medium for rapid wound healing. Further advantages are the inconspicuity in use, secure adherence, absorption of exudate (from a blister for example), good cushioning and painless removability.

Typical compositions of the first commercially available hydrocolloids for wound management products comprise:

Low molecular weight polyisobutylene (40% by weight).

Pectin (20% by weight),

Sodium carboxymethylcellulose=CMC (20% by weight)

Gelatin (20% by weight)

Modern hydrocolloid formulations, known as integrated formulations, as available for example from Coloplast, are based on styrene-isoprene-styrene block polymers with hydrocarbon resins as tackifiers, mineral oil as a plasticizer and also CMC as an absorbent. These formulations with SIS as scaffold formers contain glassy domains (styrene blocks) and thermoplastic domains (isoprene blocks). At room temperature, the glassy domains provide a kind of three-dimensional crosslinking structure, which disappears at higher temperature. Hydrocolloids possess good wet tack even under moist conditions, so that they are very useful as blister plasters on the heel and palm of the hand, an application site involving a relatively high moisture loss from the skin.

EP 0 264 299 B1 discloses a dressing consisting of a water absorbent sealing pad which in turn is formed of one or more hydrocolloids. The hydrocolloid or hydrocolloids are dissolved in or mixed with a binder. The pad is firmly and completely enclosed by a watertight cover layer. According to the invention, the pad is beveled at least along the outer periphery such that the thickness at the edge does not exceed about one quarter of its maximum thickness. It is manufactured by diecasting at high pressures and high temperatures. This process is unsuitable for crosslinked polymer gels for example polyurethane gels.

WO 92/05755 discloses contoured wound contact materials comprising an adhesive layer consisting of swellable hydrocolloids and water-insoluble, viscous constituents, for example polyisobutylene, rubber, silicone or polyurethane elastomers. The layer of adhesive material in the edge region, which is of the same kind as the adhesive material in the central region, has a thickness of less than 0.5 mm (preferably less than 0.3 mm) and a width of at least 5 mm (preferably at least 10 mm). The hydrocolloid-based adhesive material possesses tack even on moist ground.

Water-free hydrogels are known as xerogels and are macromolecular, natural or synthetic materials which contain a high level of hydrophilic groups and are consequently capable of absorptively binding water. The water absorption capacity of many xerogels is the multiple of their own weight in the water-free state. Hydrogels or xerogels are used in wound management in various forms, since they protect wounds against drying out, absorb wound exudate, serve as a matrix for active substances of all kinds and also as a basis for colonization with autologous or heterologous skin cells.

Gels may be used in the form of foams, inter alia. Foams for managing skin wounds or surgical wounds are known per se to one skilled in the art. Polyurethane foams or collagen foams are used in the main.

Self-adhesive gel foams are likewise known to one skilled in the art. They are generally readily fixable on the skin, but usually have the disadvantage that their water absorption capacity and their water retention capacity are substantially limited.

Also known are hydrophilic foams of polyurethane gels. WO 88/01878 A1 describes self-adhesive polyurethane foams or polyurethane foam gels which can contain copolymerized methacrylate units among others. These foam gels are produced by adding water.

Polyurethane gels based on a polyurethane matrix and high molecular weight polyols are also described in EP 0 057 839 B1. Self-adhesive sheet materials comprising polyurethane gels are known from EP 0 147 588 B1. The polyurethane gels disclosed in these last two references cited are unfoamed. The self-adhesive gels have isocyanate indexes of 15 to 70 (EP 0 147 588 A2).

EP 0 196 364 A2 describes hydrophilic polyurethane foams which may be filled with water absorbent polymers based on a copolymer of acrylic acid and potassium acrylate and are intended for medical purposes. The polyurethane is prepared on the basis of MDI. The polyether used has a minimum functionality of two hydroxyl groups, preferably two to three hydroxyl groups in each case. The NCO/OH ratio is stoichiometric. The polyurethane is accordingly not gellike. It can be foamed with pressurized air or with other gases which do not react with isocyanate or by means of low-boiling solvents. Absorbent and polyetherpolyol are mixed in a ratio of about 3:1. The foam has adhesive properties on wounds, which have to be completely eliminated by means of an aluminized veil in order that the foam may be used for wound treatment.

Foam wound contact materials as obtainable for example from Beiersdorf under the name of Cutinova® thin and Cutinova® hydro are described inter alia in DE 42 33 289 A1, in DE 196 18 825 A1 and WO 97/43328.

According to these references, the polyurethane gel foam consists of a polyaddition product of a polyetherpolyol (Levagel® from Bayer AG) with an aromatic or aliphatic diisocyanate (Desmodur® Bayer AG), into which a polyacrylate superabsorbent powder (Favor®, Stockhausen) has been incorporated. The polyurethane gel can be made weakly or strongly self-adherent to skin depending on the ratio of OH equivalents of the polyol to reactive isocyanate groups.

The sheetlike polyurethane gel foam from 1 to 6 mm in thickness is covered by a polyurethane film on one side. Plasters of appropriate size are punched out of the bale material. The wound contact material thus produced surprisingly abheres completely on absorption of wound fluid and, in the process, does not show the tendency, known from hydrocolloids, to disintegrate on pronounced swelling, which may lead to residues of the hydrocolloid remaining in the wound. The punched-out large-area wound contact materials are very useful for managing chronic or slow-healing wounds of patients who have to be treated in hospital.

In the case of smaller minor injuries or blistering due to pressure points on the hand, ball and heel, however, this product construction has some disadvantages. The product tends to curl under mechanical loading, owing to its punching edges. The open punching edges prove to be a disadvantage on contact with moisture, since they provide a way for water to get into the absorbent layer and cause swelling and abhering of the polyurethane gel by penetration of moisture from the side. Furthermore, the significant product height (up to 4 mm) and the same self-adhesive properties at the edge favor the adhesion of soil and curling due to adhesion to garments for example.

EP 0 680 299 A1 describes a process for the preparation of a dressing which is thin at the edges and consists of at least two layers of adhesive material. The adhesive layers, which can be of the same or of a different kind, correspond to an arrangement of mutually connected different areas which decrease in their size toward the peak. The individual layers have a step profile which, to obtain a continuous external profile, has to be covered with a further layer. A further disadvantage is the stepwise flattening of the dressing on the wound facing side, so that as a result contact is nonuniform in some areas in the wound and wound edge region.

EP 0 919 211 A2 discloses the manufacture of wound dressings having beveled edges from thermoformed polymeric carrier films which are release coated and have a cavity into which a self-adhesive, hydrophilic polymer gel is introduced. The dressings have an adhesive cover layer which in turn is covered by a protective layer. The process is cumbersome and unsuitable for wound dressings comprising a unified whole. The dressings are beveled on the wound facing side here too.

Finally, wound management additionally utilizes traditional plasters (for example the Hansaplast® classic fabric plaster from Beiersdorf) which are only contingently useful as blister plasters for the care of pressure points or damaged horny skin on highly contoured parts of the body. Disadvantages prove to be the low elasticity and the tendency for the carrier material to curl up at the edges of the plaster when subjected to mechanical loading in the course of prolonged wear. Additionally, the plaster is substantially wetted through in the course of daily ablutions or hand washing and loses adhesion. Traditional plasters are visually very conspicuous, hinder movements and impair the wear comfort in shoes.

It is an object of the present invention to provide a wound dressing which is capable of absorbing exudate from wounds, which provides good cushioning, which has a sufficient transmission rate for moisture from the skin through the plaster to the outside and which creates a moist wound healing medium.

This object is achieved by a wound dressing as defined in claim 1 and claim 3. The subclaims encompass advantageous variants of the subject matter of the invention.

Accordingly, the invention provides a wound dressing comprising a moisture vapor pervious polyurethane matrix beveled from a central point of the wound dressing toward the edge.

More particularly, the point is situated at the area's midpoint in order that a symmetrical appearance may be obtained for the plaster. But the beveling may also be uneven, depending on the requirements and application scenario of the plaster. This results in a very wide variety of shapes. The matrix may for example have a lenticular or semispherical shape.

In a preferred embodiment of the subject matter of the invention, the moisture vapor pervious polyurethane matrix has been applied uniformly atop a carrier film.

The inventive concept further comprehends a wound dressing comprising a carrier film atop which an adhesive layer has been applied uniformly, wherein there is disposed in the center of the adhesive layer a moisture vapor pervious polyurethane matrix, although the periphery of the adhesive layer is at least partially not covered by the polyurethane matrix.

The polyurethane matrix in the central zone occupies at least 1% to not more than 99% of the total area of the product.

In a further preferred embodiment, the polyurethane matrix is beveled toward the edge of the adhesive layer.

The height of the edge zone is not more than 50% of the overall height of the product, preference being given to edge zone heights of less than 0.2 mm. The contouring profile from the midpoint to the edge is defined by the mold chosen, i.e., the casting mold determines the design of the contour.

In a further preferred embodiment, the especially flexible, moisture vapor pervious or occlusive carrier film comprises polyurethane, polyethylene, polypropylene, polyamide or polyester and/or is between 60 and 80 μm in thickness. But this enumeration does not claim to be complete; it will be appreciated that one skilled in the art may come up with other suitable films without an inventive step.

The adhesive layer, preferably from 35 to 50 μm in thickness, must be considered thin. In a further advantageous embodiment, it comprises a skin-friendly pressure sensitive adhesive composition comprising polyacrylate or rubber into which a tackifier (a hydrocarbon resin for example) can be incorporated to enhance the adhesion to the skin. But this enumeration does not claim to be complete; it will be appreciated that one skilled in the art may come up with other suitable adhesive materials without an inventive step.

If appropriate, the self-adhesive product is covered with a siliconized paper or an especially siliconized film, so that the adhesive side is protected during storage.

In a further preferred embodiment, the polyurethane matrix contains one or more active substances.

Typical active substances are for the purposes of the present invention, without wishing to claim completeness:

| Indication: | Active substance |
|---|---|
| Antimycotics | naftifine |
| | amorolfine |
| | tolnaftate |
| | ciclopirox |
| Antiseptics | thymol |
| | eugenol |
| | triclosan |
| | hexachlorophene |
| | benzalkonium chloride |
| | clioquinol |
| | quinolinol |
| | undecenoic acid |
| | ethacridine |
| | chlorohexidine |
| | hexetidine |
| | dodicine |
| | iodine |
| Nonsteroidal antirheumatics | glycol salicylate |
| | flufenamic acid |
| | etofenamate |
| | ketoprofen |
| | piroxicam |
| | indomethacin |
| Antipruritics | polidocanol |
| | isoprenaline |
| | crotamiton |
| Local anesthetics | benzocaine |
| Antipsoriatics | ammonium bitumasulfonate |
| Keratolytics | urea |

The polyurethane material can be unfoamed, foamed, unfilled or filled with additional fillers, for example superabsorbents, titanium dioxide, zinc oxide. plasticizers, dyes, etc. For applications in the field of transdermal plaster systems, it is also possible to dope the polyurethane material with active substances in the central zone. It is also possible to use hydrogels in semisolid to solid form having active constituents for the central zone.

Useful polyurethanes for the matrix form part of the subject matter of DE 196 18 825, which discloses hydrophilic, self-adhesive polyurethane gels consisting of

a) polyetherpolyols with 2 to 6 hydroxyl groups and having OH values of 20 to 112 and an ethylene oxide (EO) content of ≧10 weight %,

b) antioxidants,

c) bismuth(III) carboxylates soluble in the polyols a) and based on carboxylic acids having 2 to 18 carbon atoms as catalysts and also

d) hexamethylene diisocyanate,

wherein the product of the functionalities of the polyurethane-forming components a) and d) is at least 5.2, the quantity of catalyst c) amounts to 0.005 to 0.25 weight %, relative to the polyol a), the quantity of antioxidants b) is in the range from 0.1 to 1.0 weight %, based on polyol a), and the ratio of free NCO groups of component d) to the free OH groups of component a) (isocyanate index) is selected within the range from 0.30 to 0.70.

Preference is given to using polyetherpolyols having 3 to 4, most preferably 4, hydroxyl groups and an OH number in the range from 20 to 112, preferably 30 to 56. The ethylene oxide content in the polyetherpolyols used according to the invention is preferably ≧20% by weight.

Polyetherpolyols are known per se and are prepared for example by polymerization of epoxides, such as ethylene oxide, propylene oxide, butylene oxide or tetrahydrofuran, with themselves or by addition of these epoxides, preferably of ethylene oxide and propylene oxide—optionally mixed with each other or separately in succession—to starter components having at least two reactive hydrogen atoms, such as water, ethylene glycol, propylene glycol, diethylene glycol, dipropylene glycol, glycerol, trimethylolpropane, pentaerythritol, sorbitol or sucrose. Representatives of the useful high molecular weight polyhydroxy compounds mentioned are recited for example in High Polymers, Vol. XVI, “Polyurethanes, Chemistry and Technology” (Saunders-Frisch, Interscience Publishers, New York, volume 1, 1962, pages 32-42).

The isocyanate component used is monomeric or trimerized hexamethylene diisocyanate or hexamethylene diisocyanate modified by biuret, uretidione, allophanate groups or by prepolymerization with polyetherpolyols or mixtures of polyetherpolyols based on the known starter components with 2 or >2 reactive hydrogen atoms and epoxides, such as ethylene oxide or propylene oxide having an OH number of ≦850, preferably 100 to 600. Preference is given to the use of modified hexamethylene diisocyanate, especially hexamethylene diisocyanate modified by prepolymerization with polyetherdiols of OH number 200 to 600. Very particular preference is given to modifications of hexamethylene diisocyanate with polyetherdiols of OH number 200-600 whose residual level of monomeric hexamethylene diisocyanate is below 0.5% by weight.

Useful catalysts for the polyurethane gels of the invention are bismuth(III) carboxylates which are soluble in the water-free polyetherpolyols a) and are based on linear, branched, saturated or unsaturated carboxylic acids having 2 to 18, preferably 6 to 18, carbon atoms. Preference is given to Bi(III) salts of branched saturated carboxylic acids having tertiary carboxyl groups, such as 2,2-dimethyloctanoic acid (for example Versatic acids, Shell). Of particular suitability are formulations of these Bi(III) salts in excess fractions of these carboxylic acids. Of outstanding utility is a solution of 1 mol of the Bi(III) salt of Versatic 10 acid (2,2-dimethyloctanoic acid) in an excess of 3 mol of this acid with a Bi content of about 17%.

The catalysts are preferably used in amounts from 0.03 to 0.1% by weight, based on polyol a).

Useful antioxidants for the polyurethane gels of the invention are in particular sterically hindered phenolic stabilizers, such as BHT (2,6-ditert-butyl-4-methylphenol), Vulkanox BKF (2,2′-methylenebis(6-tert-butyl-4-methylphenol) (Bayer AG), Irganox 1010 (pentaerythrityl tetrakis-[3-(3,5-ditert-butyl-4-hydroxyphenyl)propionate]), Irganox 1076 (octadecyl 3(3,5-ditert-butyl-4-hydroxyphenyl)propionate) (Ciba-Geigy) or tocopherol (vitamin E). Preference is given to using those of the α-tocopherol type.

The antioxidants are preferably used in amounts from 0.15 to 0.5% by weight, based on polyol a).

The isocyanate index (ratio of the free NCO groups used in the reaction to the free OH groups) of the polyurethane gel compositions according to the invention is in the range from 0.30 to 0.70, preferably in the range from 0.45 to 0.60, depending on the functionality of the isocyanate and polyol components used. The isocyanate index required for gel formation is very simple to estimate by the following formula:

ƒ_{(polyol)}•(ƒ_{(isocyanate)}−1)•index≈2

$$index \approx \frac{2}{{f}_{( polyol )} \cdot ( {f}_{( isocyanate )} - 1 )}$$

f: functionality of isocyanate or polyol component

Depending on the tackiness or elasticity required of the gel, the isocyanate index to be actually used may differ from the calculated value by up to +20%. The polyurethane gel compositions of the invention are prepared by customary processes as described for example in Becker/Braun, Kunststoff-Handbuch, volume 7, Polyurethane, pages 121 ff, Carl-Hauser, 1983.

Preference is further given to the use of polyurethanes as disclosed in EP 0 665856 B1. The hydrophilic polyurethane gel foams are accordingly obtainable from

1. a polyurethane gel which comprises

(A) 25-62% by weight, preferably 30-60% by weight, particularly preferably 40-57% by weight, based on the sum total of (A) and (B), of a covalently crosslinked polyurethane as a high molecular weight matrix and

(B) 75-38% by weight, preferably 70-40% by weight, particularly preferably 60-43% by weight, based on the sum total of (A) and (B), of one or more polyhydroxy compounds which are firmly held in the matrix by secondary valence forces and have an average molecular weight between 1000 and 12,000, preferably between 1500 and 8000, particularly preferably between 2000 and 6000, and an average OH number between 20 and 112, preferably between 25 and 84, particularly preferably between 28 and 56, as a liquid dispersant, the dispersant being substantially free of hydroxy compounds having a molecular weight below 800, preferably below 1000, particularly preferably below 1500, and optionally

(C) 0 to 100% by weight, based on the sum total of (A) and (B), of filler and/or additive substances,

and which is obtainable by reaction of a mixture of

a) one or more polyisocyanates,

b) one or more polyhydroxy compounds having an average molecular weight between 1000 and 12,000 and an average OH number of between 20 and 112,

c) optionally catalysts or accelerants for the reaction between isocyanate groups and hydroxyl groups and also optionally

d) filler and additive substances known per se from polyurethane chemistry,

wherein this mixture is substantially free of hydroxy compounds having a molecular weight below 800, the average functionality of the polyisocyanates (F_{I}) is between 2 and 4, the average functionality of the polyhydroxy compound (Fₚ) is between 3 and 6 and the isocyanate index (K) conforms to the formula

$$K = \frac{300 \pm X}{( {F}_{l} \cdot {F}_{p} ) - 1} + 7$$

where X is ≦120, preferably X is ≦100, particularly preferably X is ≦90, and the K index has values between 15 and 70, the specified molecular weight and OH number averages being number averages,

2. a water absorbent material and

3. a nonaqueous foaming agent.

The polyurethane gels are preparable from the starting compounds known per se from polyurethane chemistry by processes known per se as described for example in DE 31 03 499 A1, DE 31 03 500 A1 and EP 0 147 588 A1. It is essential, however, that the above-defined conditions be adhered to for the selection of the gel-forming components, or nontacky, elastic gels will be obtained instead of self-adhesive gels.

Preferred polyhydroxy compounds are polyetherpolyols as more particularly specified in the abovementioned laid-open specifications.

Useful polyisocyanate components include not only (cyclo)aliphatic but also aromatic isocyanates. Preferred (cyclo)aliphatic polyisocyanates are 1,6-hexamethylene diisocyanate and also its biurets and trimers and hydrogenated diphenylmethane diisocyanate (“MDI”) grades. Preferred aromatic polyisocyanates are those which are obtained by distillation, such as MDI mixtures of 4,4′- and 2,4′-isomers or 4,4′-MDI, and also toluylene diisocyanate (“TDI”) grades.

The diisocyanates can be selected in particular for example from the group of the unmodified aromatic or aliphatic diisocyanates or else from modified products formed by prepolymerization with amines, polyols or polyetherpolyols.

The polyurethane gels may optionally contain additives known per se from polyurethane chemistry, for example fillers and short fibers based on inorganics or organics, metal pigments, surface-active substances or liquid extenders such as substances having a boiling point of above 150° C. Useful organic fillers include for example barite, chalk, gypsum, kieserite, sodium carbonate, titanium dioxide, cerium oxide, quartz sand, kaolin, carbon black and microballoons. Useful organic fillers include for example powders based on polystyrene, polyvinyl chloride, urea-formaldehyde and polyhydrazodicarbonamide. Useful short fibers include for example glass fibers 0.1-1 mm in length or fibers of organic origin such as for example polyester or polyamide fibers. Metal powders, for example iron or copper powder, can likewise also be used in gel formation. To confer the desired color on the gels, it is possible to use the organic or inorganic dyes or color pigments known per se for the coloration of polyurethanes, for example iron oxide or chromium oxide pigments, phthalocyanine- or monoazo-based pigments. Useful surface-active substances include for example cellulose powder, active carbon and silica products.

To modify the adhesive properties of the gels, they may optionally include adds of polymeric vinyl compounds, polyacrylates and other copolymers customary in adhesive technology or else adhesives based on natural materials up to a level of 10% by weight, based on the weight of the gel composition.

Preferred water-absorbing materials are the water-absorbing salts, known as superabsorbents, of polyacrylates and copolymers thereof, especially the sodium or potassium salts. They may be crosslinked or uncrosslinked and are also obtainable as commercial products. Particularly suitable products are those disclosed in DE 37 13 601 A1 and also new-generation superabsorbents with only low remaining contents of water which can be dried out and high swelling capacity under pressure. Preferred products are lightly crosslinked polymers based on acrylic acid/sodium acrylate. Such sodium polyacrylates are obtainable as Favor (Chemische Fabrik Stockhausen GmbH, Germany). Further absorbents are likewise suitable, for example carboxymethylcellulose and karaya.

The degree of foaming can be varied within wide limits through the incorporated amounts of foaming agent.

Adhesive wound dressings which are not absorbent in the edge region comprise by virtue of their construction an advantageous combination of good product adhesion to the skin, which protects the product during use against curling and also ingress of moisture, and good absorbency for wound exudate coupled with nonadherence in the central region of the wound management product. The functionality of the product can thus be substantially extended compared with conventional products and is also not impaired through the external action of moisture (bathing, showering).

Thus protected self-adhesive and absorbent wound contact regions according to the invention are manufactured by metering and applying a defined amount of a still liquid polyurethane gel or polyurethane foam into a mold lined with a flexible carrier material having self-adhesive, nonadhesive or release properties. After the polyurethane has been applied, the outer layer is covered with a covering material (likewise with self-adhesive, nonadhesive or release properties depending on the intended use and the carrier material used) and the the polyurethane is subsequently cured to form a firm gel or foam.

It is also possible first to apply the liquid polyurethane material to a release covering material and subsequently, before curing, to effect the shaping of the polyurethane gel or polyurethane foam by applying a mold lined with a flexible carrier material having self-adhesive, nonadhesive or release properties.

Both production processes provide for the inventive production of single- or multilayered, naturally three-dimensionally contoured wound management products. The wound contact region may correspond to a conventional shape (a carrier material supported, raised, for example rectangular wound contact material or for example lenticularly curved with an outwardly convex shape or combined convex and concave elements, the wound facing surface being planar).

The latter has the advantage that, between the fixing edge layer and the wound management region, there is no longer any stepped transition to hinder the adhesion of the edge adhesive and the wound contact region which is self-adhesive to intact skin can be fully utilized as an additional fixing area, and this ultimately can lead overall to a smaller wound management product for a given size of wound.

The geometries of the contoured articles which can be produced are various (round, elliptical, square, triangular, etc.). The adhesive area consists most simply of an adhesive polyurethane material (gel or foam) as unified whole or of two concentric zones with two different adhesive materials. The central zone, which serves to absorb wound fluid or to release active substances, consists of the polyurethane gel or foam, whereas the edge zone may include various adhesive materials (filled or unfilled polyurethane, polyacrylate, rubber, SEBS adhesive material etc.), depending on the desired application and stated requirement (adhesive force etc.).

The processing time of the reactive polyurethane material is policed via the pot life, which should be between 2 and 10 minutes.

The processes described are superior to conventional processes particularly by virtue of the material saved as a consequence of the metering and processing of defined quantities.

In what follows, five illustrations and a plurality of examples are used to illustrate particularly advantageous embodiments of the dressing without thereby wishing to unnecessarily restrict the invention.

### EXAMPLES

### Production of Formed PU Articles Having Flat Edges

### Example 1

### 1 Layer Construction

Material:

| | |
|---|---|
| Casting mold: | 6 cm in diameter; 1.8 mm in depth |
| Stamping mold: | 10 cm in diameter |
| 9 g of PU composition | having an isocyanate index of 0.47 and based on a |
| | polyisocyanate (HDI)/polyetherpolyol |
| | containing |
| | 10% by weight of Favor T superabsorbent |
| | 0.4% by weight of vitamin E |
| | 0.08% by weight of Coscat 83 (Bi-based catalyst) |

Release film (siliconized PE film) and release paper

Procedure:

Place release film into the mold, meter in composition, cover with release paper, place planar plate on top and load with 2 kg weight, cure and stamp.

| | |
|---|---|
| Sample: | PU matrix; |
| Thickness in the middle: | 2.3 mm; |
| Thickness at the edge: | 0.7 mm. |

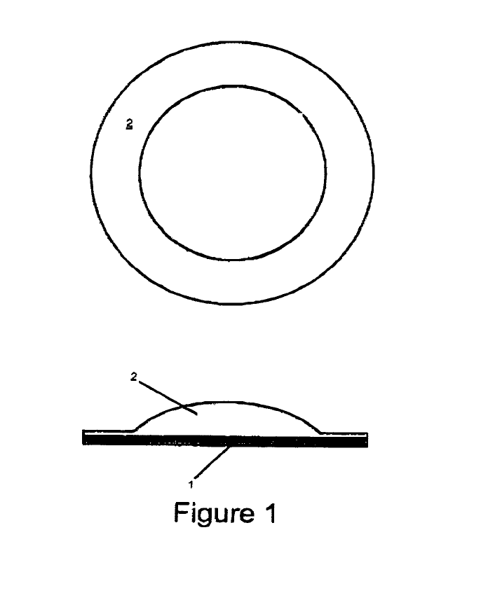

FIG. 1 illustrates a preferred geometric shape for the wound dressing, as used for blister plasters in particular.

The plaster has a circular shape (diameter 100 mm) and consists of a moisture vapor pervious polyurethane matrix 2 which is beveled toward the edge. The polyurethane matrix 2 is initially beveled uniformly and ends in a 20 mm wide ring for which the thickness is kept constant. The polyurethane matrix 2 has a substantially semiconvex shape in the middle and is accordingly comparable to a semiconvex lens.

The thickness of the polyurethane matrix 2 is 2.3 mm in the middle and 0.7 mm at the edge.

The polyurethane matrix 2 is finally covered by a siliconized paper to prevent soiling or contamination of the matrix 2.

### Example 2

### 2 Layer Construction

Material:

| | |
|---|---|
| Casting mold: | oval (25 × 46 mm); 1.3 mm in depth |
| Stamping mold; | oval (42 × 68 mm) |
| 2 g of PU composition | having an isocyanate index of 0.47 and based on a |
| | polyisocyanate (HDI)/polyetherpolyol |
| | containing |
| | 10% by weight of Favor T superabsorbent |
| | 0.4% by weight of vitamin E |
| | 0.08% by weight of Coscat 83 (Bi-based catalyst) |

Release film (siliconized PE film) and release paper

Procedure:

Place PE film into the mold, meter in composition, cover with release paper, place planar plate on top and load with 2 kg weight, cure and stamp.

| | |
|---|---|
| Sample: | PE film as covering; |
| | PU composition as wound cover and edge adhesive; |
| | Thickness in the middle: 1.6 mm; |
| | Thickness at the edge: 0.3 mm. |

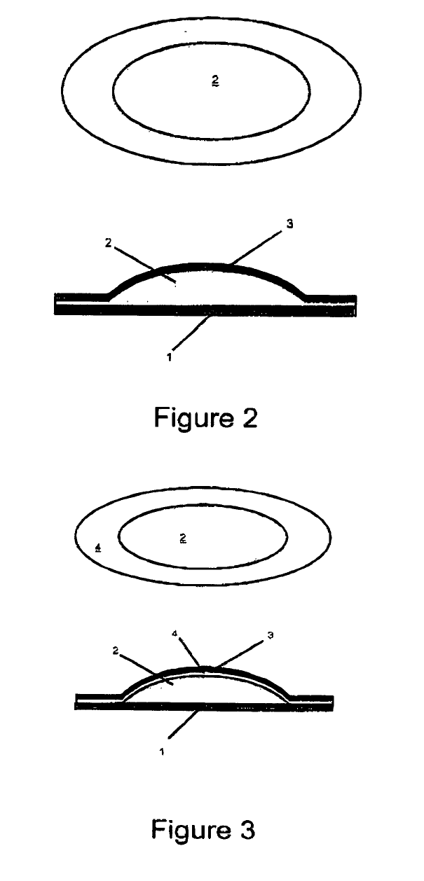

FIG. 2 illustrates a further preferred geometric shape for the wound dressing.

The plaster has an ellipsoidal shape (axes 42 mm and 68 mm in length) and consists of a moisture vapor pervious polyurethane matrix 2, which is beveled toward the edge. The polyurethane matrix 2 is initially beveled uniformly and ends in an approximately 11 mm wide ring for which the thickness is kept constant. The polyurethane matrix 2 has a substantially semiconvex shape in the middle and is accordingly comparable to a semiconvex lens.

The PU matrix 2 is covered by a PE film 3 on the skin remote side. The thickness of the polyurethane matrix 2 including the PE film 3 is 1.6 mm in the middle and 0.3 mm at the edge.

The polyurethane matrix 2 is finally covered by a siliconized paper to prevent soiling or contamination of the matrix 2.

### Examples 3.1 to 3.3

### 3 Layer Construction

### Example 3.1

Material:

| | |
|---|---|
| Casting mold: | oval (34 × 72 mm); 1.2 mm in depth |
| Stamping mold: | oval (65 × 110 mm) |
| 1.2 g of PU composition | having an Isocyanate index of 0.48 and based |
| | on a polyisocyanate (HDI)/polyetherpolyol |
| | containing |
| | 22.5% by weight of Favor T superabsorbent |

PU film adhesified with Pu composition and filled with 20% of isopropyl palmitate (IPP); release paper

Procedure:

Place PE film into the mold, meter in composition, cover with release paper, place planar plate on top and load with 2 kg weight, cure and stamp.

| | |
|---|---|
| Sample: | PE film as covering: |
| | PU composition with superabsorbent as wound cover and PU |
| | composition with IPP as edge adhesive; |
| | Thickness in the middle; 1.3 mm; |
| | Thickness at the edge: 0.15 mm. |

FIG. 3 illustrates a further preferred geometric shape for the wound dressing.

The plaster has an ellipsoidal shape (axes 110 mm and 65 mm in length) and consists of a moisture vapor pervious polyurethane matrix 2, which is beveled toward the edge. The polyurethane matrix 2 has a substantially semiconvex shape and is accordingly comparable to a semiconvex lens having axes 72 mm and 34 mm in length.

The PU matrix 2 is covered on the skin remote side by a PE film 3 which is uniformly coated with the adhesive layer 4 which is based on polyurethane and contains IPP. In the embodiment of the plaster shown here, the entire periphery of the adhesive layer 4 is not covered by the polyurethane matrix 2. This results in two concentric zones of chemically different adhesive materials 2, 4 which differ with regard to adherence, absorptivity and cushioning.

The thickness of the polyurethane matrix 2 including the PU film 3 and the adhesive layer 4 is 1.3 mm in the middle and 0.15 mm at the edge.

The polyurethane matrix 2 is finally covered by a siliconized paper to prevent soiling or contamination of the matrix 2.

### Example 3.2

Material:

| | |
|---|---|
| Casting mold: | 6 cm in diameter; 1.8 mm in depth |
| Stamping mold: | 10 cm in diameter |
| 1.5 g of PU composition | having an isocyanate index of 0.46 and based |
| foamed, density | on a polyisocyanate (HDI)/poly- |
| 0.65 g/cm³ | etherpolyol containing |
| | 22.5% by weight of Favor T superabsorbent |

PU film adhesified with acrylate composition, release paper

Procedure:

Place PU film into the mold, meter in composition, cover with release paper, place planar plate on top and load with 2 kg weight, cure and stamp.

| | |
|---|---|
| Sample: | PU film as covering; |

PU composition, foamed, as wound cover and acrylate composition as
edge adhesive;
Thickness in the middle: 1.5 mm;
Thickness at the edge: 0.10 mm.

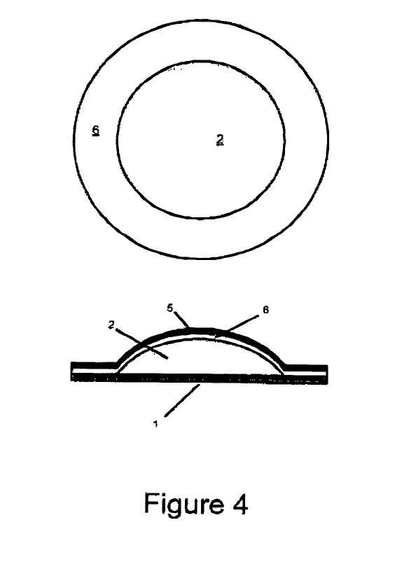

FIG. 4 illustrates a further preferred geometric shape for the wound dressing.

The plaster has an circular shape (diameter 110 mm) and consists of a moisture vapor pervious foamed polyurethane matrix 2, which is beveled toward the edge. The polyurethane matrix 2 has a substantially semiconvex shape and is accordingly comparable to a semiconvex lens having a diameter of 60 mm.

The PU matrix 2 is covered on the skin remote side by a PU film 3 which is uniformly coated with the acrylate-based adhesive layer 6. In the embodiment of the plaster shown here, the entire periphery of the adhesive layer 6 is not covered by the polyurethane matrix 2. This results in two concentric zones of chemically different adhesive materials 2, 6 which differ with regard to adherence, absorptivity and cushioning.

The thickness of the polyurethane matrix 2 including the PU film 3 and the adhesive layer 6 is 1.5 mm in the middle and 0.1 mm at the edge.

The polyurethane matrix 2 is finally covered by a siliconized paper to prevent soiling or contamination of the matrix 2.

### Example 3.3

Material:

| | |
|---|---|
| Casting mold: | 3.3 cm in diameter; 1.5 mm in depth |
| Stamping mold: | 5 × 5 cm, rounded edges |
| 0.75 g of PU composition | having an isocyanate index of 0.47 and |
| | based on a polyisocyanate (HDI)/poly- |
| | etherpolyol containing |
| | 10% by weight of Favor T superabsorbent |

PE film, skin colored, adhesified with rubber compositon;
release paper

Procedure:

Place PE film into the mold, meter in composition, cover with release paper, place planar plate on top and load with 2 kg weight, cure and stamp.

| | |
|---|---|
| Sample: | PE film as covering; |
| | PU composition as wound cover and rubber composition as edge |
| | adhesive; |
| | Thickness in the middle: 1.5 mm; |
| | Thickness at the edge: 0.10 mm. |

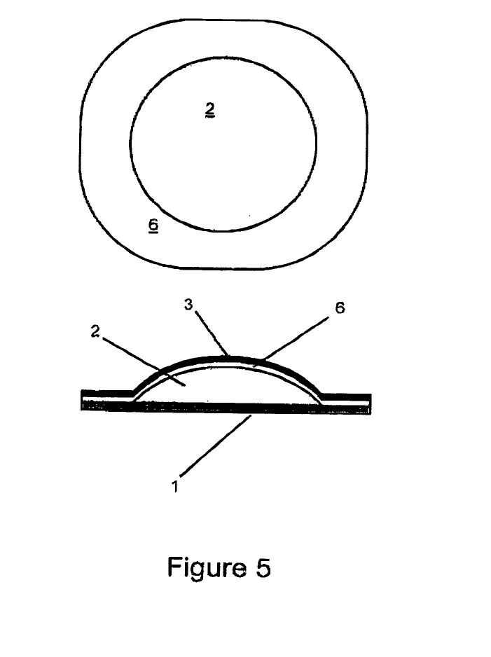

FIG. 5 illustrates a further preferred geometric shape for the wound dressing.

The plaster has a square shape, although the edges of the square are rounded (diameter of the square: 50 mm) and consists of a moisture vapor pervious foamed polyurethane matrix 2 which is beveled toward the edge. The polyurethane matrix 2 is substantially semiconvex and is circular and is accordingly comparable to a semiconvex lens having a diameter of 33 mm.

The PU matrix 2 is covered on the skin remote side by a PU film 3 which is uniformly coated with the rubber-based adhesive layer 6. In the embodiment of the plaster shown here, the entire periphery of the adhesive layer 6 is not covered by the polyurethane matrix 2. This results in two concentric zones of chemically different adhesive materials 2, 6 which differ with regard to adherence, absorptivity and cushioning.

The thickness of the polyurethane matrix 2 including the PU film 3 and the adhesive layer 6 is 1.5 mm in the middle and 0.1 mm at the edge.

The polyurethane matrix 2 is finally covered by a siliconized paper to prevent soiling or contamination of the matrix 2.

### APPENDIX

List of Inventors

1. Helge AHRENS, Alsterdorfer Strasse 371a, D-22297 Hamburg, Germany

Nationality: German

2. Michael SCHINK, Beselerstrasse 6, D-22607 Hamburg, Germany

Nationality: German

3. Jochen KENNDOFF, Jalan Bukit Dieng, Block MA 05, 65116 Malang, Jawa Timur, Indonesia

Nationality: German

4. Gunther SACHAU, Lessingstrasse 21, D-25451 Quickborn, Germany

Nationality: German

5. Reiner Leutz, Tannenallee 47, D-21485 Reinbek, Germany

Nationality: German

Claim to Priority

Priority is hereby claimed under 35 USC 119 on the basis of German Application 100 47 884.0, filed on Sep. 22, 2000. A certified copy of the German priority document will follow.

Assignment

This application is assigned to Beiersdorf Aktiengesellschaft.

What is claimed is:

1. A wound dressing comprising
a moisture vapor pervious polyurethane matrix beveled from an especially central point of the wound dressing toward the edge.

2. The wound dressing of claim 1, wherein the moisture vapor pervious polyurethane matrix has been applied uniformly atop a carrier film.

3. A wound dressing comprising
a carrier film atop which an adhesive layer has been applied uniformly, wherein there is disposed in the center of the adhesive layer a moisture vapor pervious polyurethane matrix, although the periphery of the adhesive layer is at least partially not covered by the polyurethane matrix.

4. The wound dressing of claim 3, wherein the polyurethane matrix is beveled from an especially central point of the wound dressing toward the edge of the adhesive layer.

5. The wound dressing of any of claims 1 to 4, wherein the carrier film comprises polyurethane, polyethylene, polypropylene, polyamide or polyester.

6. The wound dressing of any of claims 1 to 5, wherein the carrier film is between 60 and 80 μm in thickness.

7. The wound dressing of any of claims 1 to 6, wherein the adhesive layer comprises polyacrylate or rubber.

8. The dressing of any of claims 1 to 6, wherein the adhesive layer is between 35 and 50 μm in thickness in the edge region in particular.

9. The wound dressing of at least one preceding claim, covered over its entire width with an adhesive rejecting carrier material, such as siliconized paper, until use.

10. The wound dressing of at least one preceding claim, wherein the polyurethane matrix contains one or more active substances.
